SEQUENCE LISTING (15 sequences)

SEQ ID NO: 1 - protein, 333 aa, homo sapiens
MRIWWLLLAIEICTGNINSQDTCRQGHPGIPGNPGHNGLPGRDGRDGAKGDKGDAGEPGRPGSPGKDGTSGEKGERGADGKVEAKGIKGDQGSRGSPGKHGPKGLAGPMGEKGLRGETGPQGQKGNKGDVGPTGPEGPRGNIGPLGPTGLPGPMGPIGKPGPKGEAGPTGPQGEPGVRGIRGWKGDRGEKGKIGETLVLPKSAFTVGLTVLSKFPSSDMPIKFDKILYNEFNHYDTAAGKFTCHIAGVYYFTYHITVFSRNVQVSLVKNGVKILHTKDAYMSSEDQASGGIVLQLKLGDEVWLQVTGGERFNGLFADEDDDTTFTGFLLFSSP

SEQ ID NO: 2 - DNA, 21 bp, artificial sequence
  synthetic
cccagatgcacccattaaatt

SEQ ID NO: 3 - DNA, 27 bp, artificial sequence
  synthetic
tcaagcgtagtctgggacgtcgtatgg

SEQ ID NO: 4 - DNA, 24 bp, artificial sequence
  synthetic
cgtgacattaaggagaagctgtgc

SEQ ID NO: 5 - DNA, 24 bp, Artificial sequence
  synthetic
ctcaggaggagcaatgatcttgat

SEQ ID NO: 6 - DNA, 19 bp, artificial sequence
  synthetic
tcttttcctcggagcatga

SEQ ID NO: 7 - DNA, 23 bp, Artificial sequence
  synthetic
gacctctctactcacttctccag

SEQ ID NO: 8 - DNA, 23 bp, artificial sequence
  synthetic
agggtttagttttgagttgacgg

SEQ ID NO: 9 - DNA, 21 bp, artificial sequence
  synthetic
ccccgcttttgtcatattccg

SEQ ID NO: 10 - DNA, 20 bp, artificial sequence
  synthetic
gccgactaaatcaagcaaca

SEQ ID NO: 11 - DNA, 20 bp, artificial sequence
  synthetic
caatgggcataaagctatgg

SEQ ID NO: 12 - DNA, 20 bp, artificial sequence
  synthetic
catttattatcgcggcccta

SEQ ID NO: 13 - DNA, 20 bp, artificial sequence
  synthetic
tgttgggttgtttgatcctg

SEQ ID NO: 14 - DNA, 20 bp, artificial sequence
  synthetic
gcaattattccccatgaacg

SEQ ID NO: 15 - DNA, 20 bp, artificial sequence
  synthetic
ggcctcactaaaccatccaa

(19) United States
(12) Patent Application Publication
(10) Pub. No.: US 2015/0099695 A1
(43) Pub. Date: Apr. 9, 2015

(54) C1q/TNF-RELATED PROTEIN-9 (CTRP9) AND USE IN PREVENTION AND TREATMENT OF METABOLIC DISORDERS

(71) Applicant: THE JOHNS HOPKINS UNIVERSITY, Baltimore, MD (US)

(72) Inventors: Guang William Wong, Lutherville, MD (US); Jonathan M. Peterson, Parkville, MD (US)

(21) Appl. No.: 14/403,306

(22) PCT Filed: May 28, 2013
(86) PCT No.: PCT/US2013/042877

§ 371 Date: Nov. 24, 2014

Related U.S. Application Data
(60) Provisional application No. 61/651,696, filed on May 25, 2012.

(51) Int. Cl. C07K 14/47; A61K 45/06; C12Q 1/68; A61K 38/17

(52) U.S. Cl. CPC C07K 14/47; A61K 38/1709; A61K 45/06; C12Q 1/6883; C12Q 2600/136; C12Q 2600/158
USPC 514/4.8; 514/6.9; 514/21.2; 514/7.4; 435/6.12

(57) ABSTRACT

Compositions and methods for treatment of obesity and/or Type II diabetes and related metabolic disorders are provided wherein the methods and treatments comprise an effective amount of an isolated and/or purified C1q/TNF-related Protein-9 (CTRP9) or a functional portion thereof, and a pharmaceutically acceptable carrier. Methods of screening for molecules which elevate levels of CTRP9 in vivo are also provided. The present inventors provide the first in vivo evidence linking CTRP9 to regulation of fat metabolism in liver and skeletal muscle via AMPK signaling pathway, and highlight its protective metabolic function in the context of HFD-mediated metabolic insults.

### REFERENCE TO RELATED APPLICATIONS

This application claims the benefit of U.S. Provisional Patent Application No. 61/651,696, filed on May 25, 2012, which is hereby incorporated by reference for all purposes as if fully set forth herein.

### STATEMENT OF GOVERNMENTAL INTEREST

This invention was made with U.S. government support under grant no. R01 DK084171 and F32DK084607. The U.S. government has certain rights in the invention.

### BACKGROUND OF THE INVENTION

Adipose tissue secretes large numbers of polypeptide hormones and cytokines (collectively termed adipokines) that have local and/or systemic effects on carbohydrate and fat metabolism. These secreted metabolic regulators modulate whole body insulin sensitivity and energy metabolism by acting directly on metabolic tissues (e.g., skeletal muscle and liver) or indirectly through regulatory roles in inflammatory processes. The list of adipokines has grown considerably over the past ten years, with leptin and adiponectin being the most widely studied and best understood.

Despite the pleiotropic function of adiponectin targeted disruption of the gene in mice results in surprisingly mild metabolic phenotypes. While enhanced leptin sensitivity may partly compensate for the loss of adiponectin, other secreted factors that share overlapping function with adiponectin may play a role in ameliorating the metabolic dysfunction of adiponectin-null mice. In fact, adiponectin belongs to the C1q family of proteins, which currently consists of over 30 members, all of which possess the signature globular C1q domain. On the basis of shared sequence homology, the inventors recently identified and characterized a family of fifteen novel secreted proteins of the C1q family, designated as C1q/TNF-related proteins (CTRP1-15). Several of the CTRPs play roles in regulating glucose and fatty acid metabolism in vitro and/or in vivo.

Of the CTRPs, CTRP9 shares the highest degree of sequence identity (54%) with adiponectin at the presumed functional globular domain. In addition, CTRP9 and adiponectin share multiple common biochemical and structural features. These include adipose-selective expression, domain structure, formation of trimers, and posttranslational modifications (proline hydroxylation and lysine glycosylation). Intriguingly, CTRP9 and adiponectin also form heterotrimeric complexes in vitro and in vivo; however, the significance of this phenomenon remains unknown.

Recent works have shown that CTRP9 hormone plays a protective role in the heart against ischemia/reperfusion injury, as well as attenuating neointima formation in response to vascular injury. However, the potential protective function and mechanism of action of CTRP9 in the context of diet-induced obesity and Type II diabetes remain unknown.

There still exists, therefore, and unmet need for understanding the role that adipokines such as CTRP9 play in metabolism and Type II diabetes, and novel methods for treating these and related disorders.

### SUMMARY OF THE INVENTION

In accordance with some embodiments the present inventors provide the first in vivo evidence linking CTRP9 to regulation of fat metabolism in liver and skeletal muscle via AMPK signaling pathway, and highlight its protective metabolic function in the context of HFD-mediated metabolic insults. Thus, CTRP9 can be considered a novel component of the metabolic network that links adipose tissue to systemic energy balance.

In accordance with an embodiment, the present invention provides a medicament for use in the treatment of obesity and/or Type II diabetes in a subject comprising an effective amount of an isolated and/or purified C1q/TNF-related Protein-9 (CTRP9) or a functional portion thereof, and a pharmaceutically acceptable carrier.

In accordance with another embodiment, the present invention provides a method for treating obesity and/or Type II diabetes in a subject comprising administering to the subject an effective amount of CTRP9 protein or a functional portion thereof, in a pharmaceutically acceptable carrier.

In accordance with a further embodiment, the present invention provides a method for increasing the levels of fatty acid oxidation in the skeletal muscle of a subject comprising administering to the subject an effective amount of CTRP9 protein or a functional portion thereof, in a pharmaceutically acceptable carrier.

In accordance with yet another embodiment, the present invention provides a method for decreasing hepatic lipid accumulation in a subject comprising administering to the subject an effective amount of CTRP9 protein or a functional portion thereof, in a pharmaceutically acceptable carrier.

In accordance with a further embodiment, the present invention provides a method for prevention of diet induced insulin resistance in a subject comprising administering to the subject an effective amount of CTRP9 protein or a functional portion thereof, in a pharmaceutically acceptable carrier.

In accordance with an embodiment, the present invention provides a method for identifying a molecule which increases CTRP9 protein levels in a cell or population of cells capable of expressing the CTRP9 protein comprising: a) obtaining a cell or population of cells which express CTRP9 protein; b) incubating the molecule with the cell or population of cells of a); c) measuring the levels of CTRP9 expression, in the cell or population of cells of a); d) comparing the levels of CTRP9 in the cell or population of cells of a) to that of a control cell or population of cells; and e) determining that the molecule increases CTRP9 protein levels in a cell or population of cells when the protein levels of CTRP9 are greater than the control cell or population of cells.

### BRIEF DESCRIPTION OF THE DRAWINGS

FIG. 1 depicts how diet and metabolic state modulate circulating levels of CTRP9. 1A, Quantitative real-time PCR analyses of CTRP9 expression in adipose tissue isolated from 12-week-old chow-fed male mice under fasted or fasted/re-fed conditions. 1B, Quantitative Western blot analysis of CTRP9 serums levels in 12-week-old chow-fed male mice under fasted, fasted/re-fed, or ad libitum conditions. 1C-D, Quantitative real time PCR (1C) and Western blot (1D) analysis of CTRP9 mRNA and serum levels in male C57BL/6 mice fed a high-fat diet (HFD) or a low-fat diet (LFD) for 12 weeks. Values shown are mean±SEM. (n=8-10 mice per group)*p<0.05.

FIG. 2 shows the generation of CTRP9 gain-of-function mouse model. 2A, Schematic of CTRP9 transgene construct. HA epitope-tagged CTRP9 transgene is driven by the ubiquitous CAG promoter. 2B, Semi-quantitative RT-PCR analysis of CTRP9-HA transgene and β-Actin expression in mouse tissues. 2C, Western blot analysis of CTRP9 in WT and Tg mouse sera. 2D, Western blot analysis of CTRP9-HA protein in mouse tissues.

FIG. 3. CTRP9 Tg mice are resistant to HFD-induced obesity. 3A-B, Body weight gain over time between WT and Tg male mice fed a low-fat diet (LFD; 3A) or a high-fat diet (HFD; 3B). 3C, Representative images of HFD-fed WT and Tg mice. 3D-E, Percent fat mass (3D) and lean mass (3E) in WT and Tg mice as determined by NMR analysis. Data shown are mean±SEM (n=8-10 mice per group)*p<0.05 vs. WT.

FIG. 4 depicts reduced adiposity and adipocyte size in CTRP9 Tg mice. 4A, Quantification of subcutaneous (inguinal) fat pad mass in WT and Tg mice. 4B, Representative tissue sections of inguinal fat pad of WT and Tg mice. 4C, Quantification of visceral (gonadal) fat pad mass in WT and Tg mice. 4D, Representative tissue sections of gonadal fat pad of WT and Tg mice. Values shown are mean±SEM (n=8-10 mice per group)*p<0.05 vs. WT.

FIG. 5 shows enhanced fat oxidation and energy expenditure in CTRP9 Tg mice. 5A, Food intake analysis in WT and Tg mice on an LFD or an HFD. 5B, 24-hour ambulatory activity of WT and Tg mice on an HFD. Data were binned into 2-hour segments. 5C-F, Oxygen consumption (VO₂; 5C), carbon dioxide release (VCO₂; 5D), respiratory exchange ratio (RER=VCO₂NO₂; 5E), and energy expenditure (5F) of WT and Tg mice on a HFD as determined by indirect calorimetry. Values shown are mean±SEM (n=8 mice per group) *p<0.05 vs. WT.

FIG. 6 depicts increased mitochondrial content and expression of fat oxidation genes in skeletal muscle of Tg mice. 6A-B, Quantitative real-time analysis of fat oxidation enzyme genes (6A) and mitochondrial genes (6B) in the skeletal muscles of WT and Tg mice fed a HFD. 6C, Quantitative Western blot analysis of mitochondrion-specific protein COX IV in WT and Tg mice. COX IV levels were normalized to GAPDH protein levels. 6D, Triglyceride (TG) content in the skeletal muscles of WT and Tg mice. Values shown are mean±SEM (n=8-10 mice per group)*p<0.05 vs. WT. LCAD, long-chain acyl-CoA dehydrogenase; MCAD, medium-chain Co-A dehydrogenase; COX II, cytochrome oxidase subunit II; CytoB, mitochondria cytochrome B; COX IV, cytochrome oxidase subunit IV.

FIG. 7 shows that CTRP9 activates AMPK signaling in vivo and in vitro. 7A, Quantitative Western blot analysis of AMPKα (Thr-172) phosphorylation in the skeletal muscle of WT and Tg mice fed an HFD. Phospho-protein levels were normalized to total AMPKα levels. 7B, Western blot analysis of AMPKα phosphorylation in rat L6 myotubes stimulated with vehicle control or recombinant CTRP9 (5 μg/ml). 7C, Fatty acid (palmitate) oxidation was measured in L6 myotubes treated with vehicle control or recombinant CTRP9 (5 μg/mL). Values shown are mean±SEM. Representative gels are shown here, *p<0.05 vs. WT (n=8-10 mice per group for in vivo studies), (n=6 for in vitro experiments), representing three independent experiments.

FIG. 8 depicts reduced hepatic triglyceride accumulation in Tg mice. 8A, Representative images of WT and Tg liver sections stained with oil red O. 8B, Quantification of hepatic triglyceride (TG) levels WT and Tg mice fed an HFD. 8C, Lipid accumulation in rat H4IIE hepatocytes treated overnight with vehicle control or CTRP9 (5 μg/mL) in the presence or absence of 100 μM palmitate. All data shown are mean±SEM (n=8-10 mice per group for in vivo studies); (n=6 in vitro experiments), representing three independent experiments. Values shown are mean±SEM (n=8-10 mice per group)*p<0.05 vs. WT.

FIG. 9 depicts improved insulin sensitivity in CTRP9 Tg mice. 9A, Intraperitoneal glucose tolerance test (GTT). 9B, Quantification of the cumulative glucose clearance in GTT by integration of area under the curve (AUC). 9C, Insulin levels during the course of GTT. 9D, Quantification of the cumulative insulin release in GTT by integration of AUC. All mice were 13 weeks old and had been on HFD for the previous 9 weeks. Values are means±SEM (n=8-10 mice per group)*p<0.05 vs. WT.

### DETAILED DESCRIPTION OF THE INVENTION

As shown in the following examples, the inventors provide in vivo evidence that CTRP9 is a bona fide adipokine with important metabolic function. Transgenic mice with an elevated circulating level of CTRP9 have a remarkable ability to handle HFD-mediated metabolic challenge. These mice are resistant to HFD-induced weight gain, the development of insulin resistance, and hepatic steatosis. Reduced food intake partially accounts for the differences in body weight between Tg and WT mice in response to HFD. The substantially improved metabolic profiles of Tg mice are due to reduced adiposity and enhanced basal metabolism resulting from greater fat oxidation in the skeletal muscle and liver. Mechanistically, CTRP9 activates AMPK signaling to promote muscle fat oxidation. This in vivo effect appeared direct; treatment of myotubes with recombinant CTRP9 in vitro enhanced fatty acid oxidation, an effect abrogated by the AMPK inhibitor, compound C. In skeletal muscle, AMPK activation results in decreased synthesis of malonyl-CoA, an allosteric inhibitor of carnitine palmitoyltransferase (CPT). This promotes fatty acyl-CoA import into mitochondria for β-oxidation. Further, chronic activation of AMPK also increased mitochondrial biogenesis, and this likely accounted for the 2-fold increase in mitochondrion content seen in the skeletal muscle of Tg mice.

In accordance with an embodiment, the present invention provides a medicament for use in the treatment of obesity and/or Type II diabetes in a subject comprising an effective amount of an isolated and/or purified C1q/TNF-related Protein-9 (CTRP9) or a functional portion thereof, and a pharmaceutically acceptable carrier.

In an embodiment, the term “C1q/TNF-RELATED PROTEIN-9 (CTRP9)” means a novel and highly conserved paralog of adiponectin having the following amino acid sequence:

(SEQ ID NO: 1)
MRIWWLLLAIEICTGNINSQDTCRQGHPGIPGNPGHNGLPGRDGRDGA
KGDKGDAGEPGRPGSPGKDGTSGEKGERGADGKVEAKGIKGDQGSRGS
PGKHGPKGLAGPMGEKGLRGETGPQGQKGNKGDVGPTGPEGPRGNIGP
LGPTGLPGPMGPIGKPGPKGEAGPTGPQGEPGVRGIRGWKGDRGEKGK
IGETLVLPKSAFTVGLTVLSKFPSSDMPIKFDKILYNEFNHYDTAAGK
FTCHIAGVYYFTYHITVFSRNVQVSLVKNGVKILHTKDAYMSSEDQAS
GGIVLQLKLGDEVWLQVTGGERFNGLFADEDDDTTFTGFLLFSSP.

In accordance with another embodiment, the present invention provides a method for treating obesity and/or Type II diabetes in a subject comprising administering to the subject an effective amount of CTRP9 protein or a functional portion thereof, in a pharmaceutically acceptable carrier.

The term CTRP9 encompasses nucleic acid and polypeptide polymorphic variants, alleles, mutants, and fragments of CTRP9. Such sequences are well known in the art. Exemplary human CTRP9 sequences are available under the reference sequences NM_{—}178540 in the NCBI nucleotide database (nucleotide sequence) and accession number NP_{—}848635.2 (polypeptide sequence).

The terms “polypeptide,” “peptide” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers, those containing modified residues, and non-naturally occurring amino acid polymer.

The term “amino acid” refers to naturally occurring and synthetic amino acids, as well as amino acid analogs and amino acid mimetics that function similarly to the naturally occurring amino acids. Naturally occurring amino acids are those encoded by the genetic code, as well as those amino acids that are later modified, e.g., hydroxyproline, .gamma.-carboxyglutamate, and O-phosphoserine.

The term “amino acid analogs,” refers to compounds that have the same basic chemical structure as a naturally occurring amino acid, e.g., an .alpha. carbon that is bound to a hydrogen, a carboxyl group, an amino group, and an R group, e.g., homoserine, norleucine, methionine sulfoxide, methionine methyl sulfonium. Such analogs may have modified R groups (e.g., norleucine) or modified peptide backbones, but retain the same basic chemical structure as a naturally occurring amino acid. Amino acid “mimetics” refers to chemical compounds that have a structure that is different from the general chemical structure of an amino acid, but that functions similarly to a naturally occurring amino acid.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

As to amino acid sequences, one of ordinary skill in the art recognizes that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the invention. Typical conservative substitutions for one another: 1) Alanine (A), Glycine (G); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); 6) Phenylalanine (F), Tyrosine (Y), Tryptophan (W); 7) Serine (S), Threonine (T); and 8) Cysteine (C), Methionine (M) (see, e.g., Creighton, Proteins (1984)).

“Nucleic acid” or “oligonucleotide” or “polynucleotide” or grammatical equivalents used herein means at least two nucleotides covalently linked together. Oligonucleotides are typically from about 5, 6, 7, 8, 9, 10, 12, 15, 25, 30, 40, 50 or more nucleotides in length, up to about 100 nucleotides in length. Nucleic acids and polynucleotides are polymers of any length, including longer lengths, e.g., 200, 300, 500, 1000, 2000, 3000, 5000, 7000, 10,000, etc. A nucleic acid of the present invention will generally contain phosphodiester bonds, although in some cases, nucleic acid analogs are included that may have alternate backbones, comprising, e.g., phosphoramidate, phosphorothioate, phosphorodithioate, or O-methylphophoroamidite linkages (see Eckstein, Oligonucleotides and Analogues: A Practical Approach, Oxford University Press); and peptide nucleic acid backbones and linkages. Other analog nucleic acids include those with positive backbones; non-ionic backbones, and non-ribose backbones, including those described in U.S. Pat. Nos. 5,235,033 and 5,034,506, and Chapters 6 and 7, ASC Symposium Series 580, Carbohydrate Modifications in Antisense Research, Sanghui & Cook, eds. Nucleic acids containing one or more carbocyclic sugars are also included within one definition of nucleic acids. Modifications of the ribose-phosphate backbone may be done for a variety of reasons, e.g., to increase the stability and half-life of such molecules in physiological environments or as probes on a biochip. Mixtures of naturally occurring nucleic acids and analogs can be made; alternatively, mixtures of different nucleic acid analogs, and mixtures of naturally occurring nucleic acids and analogs may be made.

The nucleic acids may be single stranded or double stranded, as specified, or contain portions of both double stranded or single stranded sequence. As will be appreciated by those in the art, the depiction of a single strand also defines the sequence of the complementary strand; thus the sequences described herein also provide the complement of the sequence. Unless otherwise indicated, a particular nucleic acid sequence also implicitly encompasses conservatively modified variants thereof (e.g., degenerate codon substitutions) and complementary sequences, as well as the sequence explicitly indicated. The nucleic acid may be DNA, both genomic and cDNA, RNA or a hybrid, where the nucleic acid may contain combinations of deoxyribo- and ribo-nucleotides, and combinations of bases, including uracil, adenine, thymine, cytosine, guanine, inosine, xanthine hypoxanthine, isocytosine, isoguanine, etc. “Transcript” typically refers to a naturally occurring RNA, e.g., a pre-mRNA, hnRNA, or mRNA. As used herein, the term “nucleoside” includes nucleotides and nucleoside and nucleotide analogs, and modified nucleosides such as amino modified nucleosides. In addition, “nucleoside” includes non-naturally occurring analog structures. Thus, e.g. the individual units of a peptide nucleic acid, each containing a base, are referred to herein as a nucleoside.

Included in the scope of the invention are conjugates, e.g., bioconjugates, comprising any of the inventive polypeptides, or proteins (including any of the functional portions or variants thereof), nucleic acids, recombinant expression vectors, host cells, populations of host cells, or antibodies, or antigen binding portions thereof. Conjugates, as well as methods of synthesizing conjugates in general, are known in the art (See, for instance, Hudecz, F., Methods Mol. Biol. 298: 209-223 (2005) and Kirin et al., Inorg. Chem. 44(15): 5405-5415 (2005)).

The term “recombinant” when used with reference, e.g., to a cell, or nucleic acid, protein, or vector, indicates that the cell, nucleic acid, protein or vector, has been modified by the introduction of a heterologous nucleic acid or protein or the alteration of a native nucleic acid or protein, or that the cell is derived from a cell so modified. Thus, e.g., recombinant cells express genes that are not found within the native (non-recombinant) form of the cell or express native genes that are otherwise abnormally expressed, under expressed or not expressed at all. By the term “recombinant nucleic acid” herein is meant nucleic acid, originally formed in vitro, in general, by the manipulation of nucleic acid, e.g., using polymerases and endonucleases, in a form not normally found in nature. Similarly, a “recombinant protein” is a protein made using recombinant techniques, i.e., through the expression of a recombinant nucleic acid as depicted above.

Another embodiment of the invention further provides a host cell comprising any of the recombinant expression vectors described herein. As used herein, the term “host cell” refers to any type of cell that can contain the inventive recombinant expression vector. The host cell can be a eukaryotic cell, e.g., plant, animal, fungi, or algae, or can be a prokaryotic cell, e.g., bacteria or protozoa. The host cell can be a cultured cell or a primary cell, i.e., isolated directly from an organism, e.g., a human. The host cell can be an adherent cell or a suspended cell, i.e., a cell that grows in suspension. Suitable host cells are known in the art and include, for instance, DH5α E. coli cells, Chinese hamster ovarian cells, monkey VERO cells, COS cells, HEK293 cells, and the like. For purposes of amplifying or replicating the recombinant expression vector, the host cell is preferably a eukaryotic cell. More preferably, the host cell is a myocyte and/or pre-adipocyte. Most preferably, the host cell is a human cell.

Also provided by an embodiment of the invention is a population of cells comprising at least one host cell described herein. The population of cells can be a heterogeneous population comprising the host cell comprising any of the recombinant expression vectors described, in addition to at least one other cell, e.g., a host cell (e.g., a myocyte cell), which does not comprise any of the recombinant expression vectors, or a cell other than a myocyte, e.g., a B cell, a macrophage, a neutrophil, an erythrocyte, a hepatocyte, an endothelial cell, an epithelial cells, a pre-adipocyte cell, a brain cell, etc. Alternatively, the population of cells can be a substantially homogeneous population, in which the population comprises mainly of host cells (e.g., consisting essentially of) comprising the recombinant expression vector. The population also can be a clonal population of cells, in which all cells of the population are clones of a single host cell comprising a recombinant expression vector, such that all cells of the population comprise the recombinant expression vector. In one embodiment of the invention, the population of cells is a clonal population comprising host cells comprising a recombinant expression vector as described herein.

The host referred to in the inventive methods can be any host. Preferably, the host is a mammal. As used herein, the term “mammal” refers to any mammal, including, but not limited to, mammals of the order Rodentia, such as mice and hamsters, and mammals of the order Lagomorpha, such as rabbits. It is preferred that the mammals are from the order Carnivora, including Felines (cats) and Canines (dogs). It is more preferred that the mammals are from the order Artiodactyla, including Bovine (cows) and Swine (pigs) or of the order Perssodactyla, including Equine (horses). It is most preferred that the mammals are of the order Primates, Ceboids, or Simoids (monkeys) or of the order Anthropoids (humans and apes). An especially preferred mammal is the human.

As used herein, “antibody” includes reference to an immunoglobulin molecule immunologically reactive with a particular antigen, and includes both polyclonal and monoclonal antibodies. The term also includes genetically engineered forms such as chimeric antibodies (e.g., humanized murine antibodies) and heteroconjugate antibodies (e.g., bispecific antibodies). The term “antibody” also includes antigen binding forms of antibodies, including fragments with antigen-binding capability (e.g., Fab′, F(ab′).sub.2, Fab, Fv and rIgG. See also, Pierce Catalog and Handbook, 1994-1995 (Pierce Chemical Co., Rockford, Ill.). See also, e.g., Kuby, J., Immunology, 3.sup.rd Ed., W.H. Freeman & Co., New York (1998). The term also refers to recombinant single chain Fv fragments (scFv). The term antibody also includes bivalent or bispecific molecules, diabodies, triabodies, and tetrabodies. Bivalent and bispecific molecules are described in, e.g., Kostelny et al. (1992) J Immunol 148:1547, Pack and Pluckthun (1992) Biochemistry 31:1579, Hollinger et al., 1993, supra, Gruber et al. (1994) J Immuno 1:5368, Zhu et al. (1997) Protein Sci 6:781, Hu et al. (1996) Cancer Res. 56:3055, Adams et al. (1993) Cancer Res. 53:4026, and McCartney, et al. (1995) Protein Eng. 8:301.

As used herein, the term “diabetes” refers to a disease process derived from multiple causative factors and characterized by elevated levels of plasma glucose or hyperglycemia in the fasting state or after administration of glucose during an oral glucose tolerance test. Persistent or uncontrolled hyperglycemia is associated with increased and premature morbidity and mortality. Often abnormal glucose homeostasis is associated both directly and indirectly with alterations of the lipid, lipoprotein and apolipoprotein metabolism and other metabolic and hemodynamic disease. Therefore patients with Type II diabetes mellitus are at especially increased risk of macrovascular and microvascular complications, including coronary heart disease, stroke, peripheral vascular disease, hypertension, nephropathy, neuropathy, and retinopathy. Therefore, therapeutical control of glucose homeostasis, lipid metabolism and hypertension are critically important in the clinical management and treatment of diabetes mellitus.

There are two generally recognized forms of diabetes. In Type I diabetes, or insulin-dependent diabetes mellitus (IDDM), patients produce little or no insulin, the hormone which regulates glucose utilization. In Type II diabetes, or noninsulin dependent diabetes mellitus (NIDDM), patients often have plasma insulin levels that are the same or even elevated compared to nondiabetic subjects; however, these patients have developed a resistance to the insulin stimulating effect on glucose and lipid metabolism in the main insulin-sensitive tissues, which are muscle, liver and adipose tissues, and the plasma insulin levels, while elevated, are insufficient to overcome the pronounced insulin resistance.

Insulin resistance is not primarily due to a diminished number of insulin receptors but to a post-insulin receptor binding defect that is not yet understood. This resistance to insulin responsiveness results in insufficient insulin activation of glucose uptake, oxidation and storage in muscle and inadequate insulin repression of lipolysis in adipose tissue and of glucose production and secretion in the liver.

The available treatments for Type II diabetes, which have not changed substantially in many years, have recognized limitations. While physical exercise and reductions in dietary intake of calories will dramatically improve the diabetic condition, compliance with this treatment is very poor because of well-entrenched sedentary lifestyles and excess food consumption, especially of foods containing high amounts of saturated fat. Increasing the plasma level of insulin by administration of sulfonylureas (e.g. tolbutamide and glipizide) or meglitinide, which stimulate the pancreatic β-cells to secrete more insulin, and/or by injection of insulin when sulfonylureas or meglitinide become ineffective, can result in insulin concentrations high enough to stimulate the very insulin-resistant tissues. However, dangerously low levels of plasma glucose can result from administration of insulin or insulin secretagogues (sulfonylureas or meglitinide), and an increased level of insulin resistance due to the even higher plasma insulin levels can occur. The biguanides increase insulin sensitivity resulting in some correction of hyperglycemia. However, the two biguanides, phenformin and metformin, can induce lactic acidosis and nausea/diarrhea. Metformin has fewer side effects than phenformin and is often prescribed for the treatment of Type II diabetes.

Additional methods of treating the disease are still under investigation. New biochemical approaches that have been recently introduced or are still under development include treatment with alpha-glucosidase inhibitors (e.g. acarbose) and protein tyrosine phosphatase-1B (PTP-1B) inhibitors.

The compositions and methods of the present invention therefore have utility in the treatment of Type II diabetes and in the treatment and prevention of the numerous conditions that often accompany Type II diabetes, including metabolic syndrome X, reactive hypoglycemia, and diabetic dyslipidemia. Obesity, discussed below, is another condition that is often found with Type II diabetes that may respond to treatment with the compounds of this invention.

The following diseases, disorders and conditions are related to Type II diabetes, and therefore may be treated, controlled or in some cases prevented, by treatment with the compounds and methods of the present invention: (1) hyperglycemia, (2) low glucose tolerance, (3) insulin resistance, (4) obesity, (5) lipid disorders, (6) dyslipidemia, (7) hyperlipidemia, (8) hypertriglyceridemia, (9) hypercholesterolemia, (10) low HDL levels, (11) high LDL levels, (12) atherosclerosis and its sequelae, (13) vascular restenosis, (14) irritable bowel syndrome, (15) inflammatory bowel disease, including Crohn's disease and ulcerative colitis, (16) other inflammatory conditions, (17) pancreatitis, (18) abdominal obesity, (19) neurodegenerative disease, (20) retinopathy, (21) nephropathy, (22) neuropathy, (23) Syndrome X, (24) ovarian hyperandrogenism (polycystic ovarian syndrome), and other disorders where insulin resistance is a component.

The subject compositions and methods of the present invention are further useful in a method for the prevention or treatment of the aforementioned diseases, disorders and conditions in combination with other agents.

The compounds of the present invention may be used in combination with one or more other drugs in the treatment, prevention, suppression or amelioration of diseases or conditions for which CTRP9 or a functional portion thereof, or the other drugs may have utility, where the combination of the drugs together are safer or more effective than either drug alone. Such other drug(s) may be administered, by a route and in an amount commonly used therefor, contemporaneously or sequentially with CTRP9 or a functional portion thereof. When CTRP9 or a functional portion thereof is used contemporaneously with one or more other drugs, a pharmaceutical composition in unit dosage form containing such other drugs and CTRP9 or a functional portion thereof is preferred. However, the combination therapy may also include therapies in which CTRP9 or a functional portion thereof and one or more other drugs are administered on different overlapping schedules. It is also contemplated that when used in combination with one or more other active ingredients CTRP9 or a functional portion thereof and the other active ingredients may be used in lower doses than when each is used singly. Accordingly, the pharmaceutical compositions of the present invention include those that contain one or more other active ingredients, in addition to CTRP9 or a functional portion thereof.

Examples of other active ingredients that may be administered in combination with CTRP9 or a functional portion thereof, and either administered separately or in the same pharmaceutical composition, include, but are not limited to: (a) other dipeptidyl peptidase IV (DP-IV) inhibitors; (b) insulin sensitizers including (i) PPARγ agonists such as the glitazones (e.g. troglitazone, pioglitazone, englitazone, MCC-555, rosiglitazone, and the like) and other PPAR ligands, including PPARα/γ dual agonists, such as KRP-297, and PPARα agonists such as fenofibric acid derivatives (gemfibrozil, clofibrate, fenofibrate and bezafibrate), (ii) biguanides such as metformin and phenformin, and (iii) protein tyrosine phosphatase-1B (PTP-1B) inhibitors; (c) insulin or insulin mimetics; (d) sulfonylureas and other insulin secretagogues such as tolbutamide and glipizide, meglitinide, and related materials; (e) α-glucosidase inhibitors (such as acarbose); (f) glucagon receptor antagonists such as those disclosed in WO 98/04528, WO 99/01423, WO 00/39088, and WO 00/69810; (g) GLP-1, GLP-1 mimetics, and GLP-1 receptor agonists such as those disclosed in WO00/42026 and WO00/59887; (h) GIP and GIP mimetics such as those disclosed in WO00/58360, and GIP receptor agonists; (i) PACAP, PACAP mimetics, and PACAP receptor 3 agonists such as those disclosed in WO 01/23420; (j) cholesterol lowering agents such as (i) HMG-CoA reductase inhibitors (lovastatin, simvastatin, pravastatin, fluvastatin, atorvastatin, rivastatin, itavastatin, rosuvastatin, and other statins), (ii) sequestrants (cholestyramine, colestipol, and dialkylaminoalkyl derivatives of a cross-linked dextran), (iii) nicotinyl alcohol, nicotinic acid or a salt thereof, (iv) PPARα agonists such as fenofibric acid derivatives (gemfibrozil, clofibrate, fenofibrate and bezafibrate), (v) PPARα/γ dual agonists, such as KRP-297, (vi) inhibitors of cholesterol absorption, such as beta-sitosterol and ezetimibe, (vii) acyl CoA:cholesterol acyltransferase inhibitors, such as avasimibe, and (viii) anti-oxidants, such as probucol; (k) PPARδ agonists, such as those disclosed in WO97/28149; (1) antiobesity compounds such as fenfluramine, dexfenfluramine, phentermine, sibutramine, orlistat, neuropeptide Y5 inhibitors, and β3 adrenergic receptor agonists; (m) an ileal bile acid transporter inhibitor; and (n) agents intended for use in inflammatory conditions such as aspirin, non-steroidal anti-inflammatory drugs, glucocorticoids, azulfidine, and cyclo-oxygenase 2 selective inhibitors.

The above combinations include combinations of CTRP9 or a functional portion thereof not only with one other active compound, but also with two or more other active compounds. Non-limiting examples include combinations of CTRP9 or a functional portion thereof with two or more active compounds selected from biguanides, sulfonylureas, HMG-CoA reductase inhibitors, PPAR agonists, PTP-1B inhibitors, other DP-IV inhibitors, and anti-obesity compounds.

Likewise, compounds of the present invention may be used in combination with other drugs that are used in the treatment/prevention/suppression or amelioration of the diseases or conditions for which compounds of the present invention are useful. Such other drugs may be administered, by a route and in an amount commonly used therefor, contemporaneously or sequentially with a compound of the present invention. When a compound of the present invention is used contemporaneously with one or more other drugs, a pharmaceutical composition containing such other drugs in addition to the compound of the present invention is preferred. Accordingly, the pharmaceutical compositions of the present invention include those that also contain one or more other active ingredients, in addition to a compound of the present invention.

In accordance with a further embodiment, the present invention provides a method for increasing the levels of fatty acid oxidation in the skeletal muscle of a subject comprising administering to the subject an effective amount of CTRP9 protein or a functional portion thereof, in a pharmaceutically acceptable carrier.

In accordance with yet another embodiment, the present invention provides a method for decreasing hepatic lipid accumulation in a subject comprising administering to the subject an effective amount of CTRP9 protein or a functional portion thereof, in a pharmaceutically acceptable carrier.

In accordance with a further embodiment, the present invention provides a method for prevention of diet induced insulin resistance in a subject comprising administering to the subject an effective amount of CTRP9 protein or a functional portion thereof, in a pharmaceutically acceptable carrier.

The choice of carrier will be determined in part by the particular myonectin (CTRP9) protein, as well as by the particular method used to administer the myonectin (CTRP9) protein. Accordingly, there are a variety of suitable formulations of the pharmaceutical composition of the invention. The following formulations for parenteral, subcutaneous, intravenous, intramuscular, intraarterial, intrathecal and interperitoneal administration are exemplary and are in no way limiting. More than one route can be used to administer the myonectin (CTRP9) protein, and in certain instances, a particular route can provide a more immediate and more effective response than another route.

Formulations suitable for parenteral administration include aqueous and non-aqueous, isotonic sterile injection solutions, which can contain anti-oxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives.

Oils, which can be used in parenteral formulations, include petroleum, animal, vegetable, or synthetic oils. Specific examples of oils include peanut, soybean, sesame, cottonseed, corn, olive, petrolatum, and mineral. Suitable fatty acids for use in parenteral formulations include oleic acid, stearic acid, and isostearic acid. Ethyl oleate and isopropyl myristate are examples of suitable fatty acid esters.

Suitable soaps for use in parenteral formulations include fatty alkali metal, ammonium, and triethanolamine salts, and suitable detergents include (a) cationic detergents such as, for example, dimethyl dialkyl ammonium halides, and alkyl pyridinium halides, (b) anionic detergents such as, for example, alkyl, aryl, and olefin sulfonates, alkyl, olefin, ether, and monoglyceride sulfates, and sulfosuccinates, (c) nonionic detergents such as, for example, fatty amine oxides, fatty acid alkanolamides, and polyoxyethylenepolypropylene copolymers, (d) amphoteric detergents such as, for example, alkyl-β-aminopropionates, and 2-alkyl-imidazoline quaternary ammonium salts, and (e) mixtures thereof.

The parenteral formulations will typically contain from about 0.5% to about 25% by weight of the myonectin (CTRP9) protein in solution. Preservatives and buffers may be used. In order to minimize or eliminate irritation at the site of injection, such compositions may contain one or more nonionic surfactants having a hydrophile-lipophile balance (HLB) of from about 12 to about 17. The quantity of surfactant in such formulations will typically range from about 5% to about 15% by weight. Suitable surfactants include polyethylene glycol sorbitan fatty acid esters, such as sorbitan monooleate and the high molecular weight adducts of ethylene oxide with a hydrophobic base, formed by the condensation of propylene oxide with propylene glycol. The parenteral formulations can be presented in unit-dose or multi-dose sealed containers, such as ampoules and vials, and can be stored in a freeze-dried (lyophilized) condition requiring only the addition of the sterile liquid excipient, for example, water, for injections, immediately prior to use. Extemporaneous injection solutions and suspensions can be prepared from sterile powders, granules, and tablets.

Injectable formulations are in accordance with the invention. The requirements for effective pharmaceutical carriers for injectable compositions are well-known to those of ordinary skill in the art (see, e.g., Pharmaceutics and Pharmacy Practice, J.B. Lippincott Company, Philadelphia, Pa., Banker and Chalmers, eds., pages 238-250 (1982), and ASHP Handbook on Injectable Drugs, Trissel, 15th ed., pages 622-630 (2009)).

For purposes of the invention, the amount or dose of the myonectin (CTRP9) protein administered should be sufficient to effect, e.g., a therapeutic or prophylactic response, in the subject over a reasonable time frame. The dose will be determined by the efficacy of the particular myonectin (CTRP9) protein and the condition of a human, as well as the body weight of a human to be treated.

The dose of the myonectin (CTRP9) protein also will be determined by the existence, nature and extent of any adverse side effects that might accompany the administration of a particular myonectin (CTRP9) protein. Typically, the attending physician will decide the dosage of the myonectin (CTRP9) protein with which to treat each individual patient, taking into consideration a variety of factors, such as age, body weight, general health, diet, sex, vaccine protein to be administered, route of administration, and the severity of the condition being treated. By way of example and not intending to limit the invention, the dose of the myonectin (CTRP9) protein can be about 0.001 to about 1000 mg/kg body weight of the subject being treated/day, from about 0.01 to about 10 mg/kg body weight/day, about 0.01 mg to about 1 mg/kg body weight/day. In a preferred embodiment, the dose of the myonectin (CTRP9) protein administered is about 5-10 mg/kg/day.

Alternatively, the myonectin (CTRP9) protein can be modified into a depot form, such that the manner in which the vaccine protein is released into the body to which it is administered is controlled with respect to time and location within the body (see, for example, U.S. Pat. No. 4,450,150). Depot forms of myonectin (CTRP9) proteins can be, for example, an implantable composition comprising the vaccine proteins and a porous or non-porous material, such as a polymer, wherein the myonectin (CTRP9) protein is encapsulated by or diffused throughout the material and/or degradation of the non-porous material. The depot is then implanted into the desired location within the body and the myonectin (CTRP9) proteins are released from the implant at a predetermined rate.

With respect to the inventive method of detecting any of the myonectin (CTRP9) protein or nucleic acid molecules in a host, the sample of cells of the host can be a sample comprising whole cells, lysates thereof, or a fraction of the whole cell lysates, e.g., a nuclear or cytoplasmic fraction, a whole protein fraction, or a nucleic acid fraction.

For purposes of the inventive detecting method, the contacting can take place in vitro or in vivo with respect to the host. Preferably, the contacting is in vitro.

As used herein, the term “treatment,” or “modulation” is an approach for obtaining beneficial or desired clinical results. For purposes of this invention, beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. The term “treatment,” can also mean prolonging survival as compared to expected survival if not receiving treatment. The term “treatment,” is an intervention performed with the intention of preventing the development of a disorder or altering the pathology of a disorder. Accordingly, the term “treatment,” refers to both therapeutic treatment and prophylactic or preventative measures.

Expression of CTRP9 levels can be detected in a number of different ways. As described herein, the expression levels of the protein in a cell can be determined by probing the mRNA expressed in a cell with a probe that specifically hybridizes with a CTRP9 transcript (or complementary nucleic acid derived therefrom). Alternatively, protein can be detected using immunological methods in which a cell lysate is probed with antibodies that specifically bind to the protein.

Other cell-based assays are reporter assays can be conducted with cells that do not express the protein. Often, these assays are conducted with a heterologous nucleic acid construct that includes a promoter that is operably linked to a reporter gene that encodes a detectable product.

The data obtained from cell culture assays and animal studies of the present invention can be used to formulate a dosage range for use in humans. The dosage of such compounds lies preferably within a range of circulating concentrations that include the ED₅₀ with little or no toxicity. The dosage can vary within this range depending upon the dosage form employed and the route of administration. For any compound used in the methods of the invention, the therapeutically effective dose can be estimated initially from cell culture assays. A dose can be formulated in animal models to achieve a circulating plasma concentration range that includes the IC₅₀ (the concentration of the test compound that achieves a half-maximal inhibition of symptoms) as determined in cell culture. Such information can be used to more accurately determine useful doses in humans. Levels in plasma can be measured, for example, by high performance liquid chromatography (HPLC).

In some embodiments, assays comprising cells that express CTRP9 proteins that are treated with a molecule which is a potential agonist or stimulator compound are compared to control samples without the molecule, to examine the effect on activity. Typically, control samples, e.g., cells that express CTRP9 proteins and that are untreated with molecules of interest are assigned a relative protein activity value of 100%. Stimulation of CTRP9 protein levels is achieved when the activity value relative to the control is changed at least about 20%, at least about 50%, at least about 75-100%, or more.

The molecules tested as stimulators of CTRP9 protein levels and expression can be any small chemical compound, or a biological entity, e.g., a macromolecule such as a protein, sugar, nucleic acid or lipid. Typically, test compounds will be small chemical molecules and peptides or antibodies.

In some embodiments, the compounds/molecules tested have a molecular weight of less than 1,500 daltons, and in some cases less than 1,000, 800, 600, 500, or 400 daltons. The relatively small size of the agents can be desirable because smaller molecules have a higher likelihood of having physiochemical properties compatible with good pharmacokinetic characteristics, including oral absorption than agents with higher molecular weight. For example, agents less likely to be successful as drugs based on permeability and solubility were described by Lipinski et al. as follows: having more than 5 H-bond donors (expressed as the sum of OHs and NHs); having a molecular weight over 500; having a Log P over 5 (or M Log P over 4.15); and/or having more than 10 H-bond acceptors (expressed as the sum of Ns and Os). See, e.g., Lipinski et al. Adv Drug Delivery Res 23:3-25 (1997). Compound classes that are substrates for biological transporters are typically exceptions to the rule.

Essentially any chemical compound or molecule can be used as a potential stimulator or agonist in the assays of the present invention. Most often, compounds can be dissolved in aqueous or organic (especially DMSO-based) solutions. The assays are designed to screen large chemical libraries by automating the assay steps, which are typically run in parallel (e.g., in microtiter formats on microtiter plates in robotic assays). It will be appreciated that there are many suppliers of chemical compounds, including Sigma (St. Louis, Mo.), Aldrich (St. Louis, Mo.), Sigma-Aldrich (St. Louis, Mo.), Fluka Chemika-Biochemica Analytika (Buchs Switzerland) and the like.

Therefore, in accordance with an embodiment, the present invention provides a method for identifying a molecule which increases CTRP9 protein levels in a cell or population of cells capable of expressing the CTRP9 protein comprising: a) obtaining a cell or population of cells which express CTRP9 protein; b) incubating the molecule with the cell or population of cells of a); c) measuring the levels of CTRP9 expression, in the cell or population of cells of a); d) comparing the levels of CTRP9 in the cell or population of cells of a) to that of a control cell or population of cells; and e) determining that the molecule increases CTRP9 protein levels in a cell or population of cells when the protein levels of CTRP9 are greater than the control cell or population of cells.

### EXAMPLES

Antibodies and chemicals. Mouse monoclonal anti-FLAG M2 antibody was obtained from Sigma (St. Louis, Mo.), and rat monoclonal anti-HA (clone 3F10) antibody was obtained from Roche ( ). Rabbit antibodies recognizing phospho-AKT (Thr-308), phospho-AMPKα (Thr-172), Aid, AMPKα, and COX IV were obtained from Cell Signaling Technology. Goat anti-Actin antibody was obtained from Santa Cruz, and mouse anti-GAPDH monoclonal antibody was obtained from Novus Biologicals.

Animals. C57BL/6J mice (The Jackson Laboratory) were used to evaluate diet-induced changes in CTRP9 mRNA and circulating levels. For all other experiments, mice were bred and weaned at The Johns Hopkins University School of Medicine animal facilities. Four-week-old CTRP9 transgenic mice and WT control littermates were housed in polycarbonate cages on a 12 hour light-dark photocycle with ad libitum access to water throughout the study period. Male mice were used throughout the study. Mice were fed ad libitum a high-fat diet (HFD; 60% kcal derived from fat, Research Diets; D12492) or an isocaloric-matched low-fat diet (LFD; 10% kcal derived from fat, Research Diets; D12450B). HFD was provided for a period of 14 weeks. Blood samples were collected for serum analysis. Tissues were collected, snap frozen in liquid nitrogen, and kept at −80° C. All animal protocols were approved by the Institutional Animal Care and Use Committee of the Johns Hopkins University School of Medicine.

Generation of CTRP9 transgenic mice. The C-terminal HA epitope-tagged CTRP9 was cloned into the XhoI site of pCAGGS vector. Expression of CTRP9 transgene was driven by the ubiquitous CAG promoter, which consists of a CMV enhancer element with a chicken β-Actin promoter. The plasmid construct was digested with SalI and NotI restriction enzymes, and resulting DNA fragments (˜3.5 and 2.5 kb) were separated on a 1% agarose gel. The ˜3.5 kb linear DNA fragment containing the CAG promoter and enhancer, CTRP9-HA transgene, and the rabbit β-globin polyA adenylation signal was excised from the agarose gel, purified, and verified by DNA sequencing. Pronuclear injections were performed; several transgenic founder lines were obtained. Multiple founder lines were obtained and all showed a lean phenotype. However, only one line was maintained and expanded for phenotypic analysis. The transgenic line was maintained on a (C57BL/6×FVB) mixed genetic background. Transgene-negative littermates were used as WT control mice throughout the study.

Semi-quantitative PCR analysis. Total RNAs from mouse tissues were isolated with TRIzol® (Invitrogen). Two μg of total RNA were reverse-transcribed using Superscript II (Invitrogen). Thirty-cycle PCR was carried out using Hot Start Taq Blue polymerase (Denville); the cycling conditions were as follows: 15 seconds denaturation at 95° C., 15 seconds primer annealing at 60° C., and 45 seconds primer extension at 72° C. Primers used included the following: CTRP9-HA forward 5′-CCCAGATGCACCCATTAAATT CG-3′ (SEQ ID NO: 2) and reverse 5′-TCAAGCGTAGTCTGGGACGTCGTATGG-3′ (SEQ ID NO: 3); and β-Actin forward 5′-CGTG ACATTAAGGAGAAGCTGTGC-3′ (SEQ ID NO: 4) and reverse 5′-CTCAGGAGGAGCAATGATCTTGAT-3′ (SEQ ID NO: 5).

Mouse serum and blood chemistry analysis. Mouse serum samples were harvested by tail bleeding after overnight fast (˜16 hours), or at indicated time point. Serum samples were separated using Microvette® CB 300 (Sarstedt). Glucose concentration was determined at time of collection with a glucometer (BD bioscience). Serum samples were prepared according to manufacturer's instructions for individual assay or diluted 1:20 in SDS loading buffer (50 mM Tris-HCl, ph 7.4, 2% SDS w/v, 6% glycerol w/v, 1% 2-mercaptoethanol v/v, and 0.01% bromophenol blue w/v) and subjected to Western blot analysis. Serum/tissue triglyceride (Thermofisher), NEFA (Wako), insulin, leptin, and adiponectin (Millipore) were determined according to the protocol of the commercially available kit.

Intraperitoneal glucose and insulin tolerance tests. Separate cohorts of 8-10 13-week-old Tg and control littermates were injected intraperitoneally with glucose (1 g/kg). Animals were fasted for 6 hours prior to the glucose tolerance test. For insulin tolerance test, food was removed 2 hours prior to insulin injection. Serum samples were collected at the indicated time points shown in the Examples below.

Protein purification. Recombinant full-length mouse CTRP9, containing a C-terminal FLAG-tagged epitope, was produced in HEK 293T mammalian cells as previously described (FASEB J 23: 241-258, 2009).

Body composition analysis. Body compositions of Tg and control littermates were determined using a whole-body NMR instrument (EchoMRI, Waco, Tex.) at the metabolic phenotyping core facility at Johns Hopkins University School of Medicine. EchoMRI analysis provided values for fat mass, lean mass, and water content.

Quantitative real time PCR analysis. RNAs were isolated from tissues using Trizol® (Invitrogen) and reverse transcribed using Superscript II RNase H-reverse Transcriptase (Invitrogen) according to the manufacturer's protocol. PCR primers used included: LCAD forward 5′-TCTTTTCCTCGGAGCATGA CA-3′ (SEQ ID NO: 6) and reverse 5′-GACCTCTCTACTCACTTCTCCAG-3′ (SEQ ID NO: 7); MCAD forward 5′-AGGGTTTAGTTTTGAGTTGACGG-3′ (SEQ ID NO: 8) and reverse 5′-CCCCGCTT TTGTCATATTCCG-3′ (SEQ ID NO: 9); COXII forward 5′-GCCG ACTAAATCAAGCAACA-3′ (SEQ ID NO: 10) and reverse 5′-CAA TGGGCATAAAGCTATGG-3′(SEQ ID NO: 11); CytoB forward 5′-CATTTATTATCGCGGCCCTA-3′ (SEQ ID NO: 12) and reverse 5′-T GTTGGGTTGTTTGATCCTG-3′ (SEQ ID NO: 13); and 18 S rRNA forward, 5′-GCAATTATTCCCCATGAACG-3′ (SEQ ID NO: 14) and reverse, 5′-GGCCTCACTAAACCATCCAA-3′ (SEQ ID NO: 15). Analyses were performed on an Applied Biosystems Prism 7500 Sequence Detection System. Samples were analyzed in 25 μL reactions according to the standard protocol provided in the SyBR® Green PCR Master Mix (Applied Biosystems). All expression levels were normalized to the corresponding 18 S rRNA levels.

Indirect calorimetry. CTRP9 Tg mice and control littermates (n=7-8 per group) were used for simultaneous assessments of changes in daily body weight, energy intake (corrected for spillage), and whole-body metabolic profile in an open-flow indirect calorimeter (Oxymax, Columbus Instruments) as described previously (J Appl Physiol 107: 1006-1014, 2009). LFD- and HFD-fed mice were evaluated in separate studies. Data were collected for three days to confirm acclimation to the calorimetry chambers (stable body weights and food intakes), and data from the fourth day in the Oxymax were analyzed. Rates of oxygen consumption (VO₂, ml/kg/hr) and carbon dioxide production (VCO₂) were measured for each chamber every 16 minutes throughout the studies. Respiratory exchange ratio (RER=VCO₂/VO₂) was calculated by Oxymax software (v. 4.02) to estimate relative oxidation of carbohydrate (RER=1.0) versus fat (RER approaching 0.7), not accounting for protein oxidation. Energy expenditure was calculated as EE=VO₂×(3.815+(1.232×RER)) (Lusk G. The elements of the science of nutrition. Philadelphia London: W. B. Saunders company, 1928.), and normalized to lean body mass (Kcals/kg/hr) as recommended (Diabetes 59: 323-329, 2010). Average metabolic values were calculated within subjects, then averaged across subjects for statistical analysis by Student's t-test, with p≦0.05 indicating significant group differences.

Physical Activity. Mice (n=8 per group) were tested in open-field chambers with infrared beam arrays to detect movement (Photobeam Activity system (PAS)-Open Field, San Diego Instruments, San Diego, Calif.). Locomotor activity was monitored for 2 in 12 bins of 2-hours duration, and reported as total ambulatory activity (ambulatory-like beam break patterns in the center plus periphery).

Cell culture. GripTite™ HEK 293 cells (Invitrogen) were cultured in DMEM containing 10% fetal bovine serum (FBS), 2 mM L-glutamine, 100 units/mL penicillin, and 100 μg/mL streptomycin. Rat H4IIE hepatocytes and rat L6 myocytes were cultured in DMEM containing 10% FBS and antibiotics. L6 myotubes were differentiated as previously described (Biochem. Biophys. Res. Commun 388: 360-365, 2009). Each experiment represents the combined data from three independently performed experiments. For in vitro assay, free fatty acid (palmitate)/BSA (bovine serum albumin) conjugates were prepared as previously described (J Biol Chem 276: 14890-14895, 2001). Briefly, a 20 mM solution of free fatty acids in 0.01 M NaOH were incubated at 70° C. for 30 minutes, and fatty acid soaps were then complexed with 5% BSA in phosphate-buffered saline (PBS) at an 8:1 ratio of fatty acid to BSA. The conjugates were administered overnight to cultured H4IIE hepatocytes at concentrations indicated.

Fatty Acid Oxidation. To measure fatty acid oxidation, the protocol as described by Buzzai et al., was adapted in which oxidation of [9,10-³H]-palmitic acid results in formation of [³H]—H₂O (Oncogene 24: 4165-4173, 2005). In brief, rat L6 myotubes were incubated for 2 hours in serum-free DMEM containing 0.2% bovine serum albumin with recombinant CTRP9 (5 μg/mL) or vehicle buffer. Next, 0.2 μCi/ml [9,10-³H]-palmitic acid (Moravek Biochemical) was added to the media and incubated for 60 minutes. The [9,10-³H]-palmitate was oxidized to CO₂ and [³H]—H₂O. After incubation, the medium was transferred to a tube containing equal volume of chilled (4° C.) 10% trichloroacetic acid. Samples were mixed and incubated for 10 minutes at 4° C., then centrifuged for 30 minutes at 4° C. After centrifugation, 400 μl of the supernatant were collected and combined with 55 μl of 6N NaOH, then transferred to a Micro Bio-spin chromatography column (BioRad; Catalogue #732-6204), containing 0.5 g Dowex ion exchange resin (Sigma 217425). The [³H]—H₂O would be selectively retained by the resin while the hydrophobic, non-oxidized [9,10-³H]-palmitic acid in the supernatant would pass through the column. The bound [³H]—H₂O in the column was eluted with 1 ml dH₂O, and the elution was transferred to a liquid scintillation vial. The amount of ³H radioactivity was determined with a Beckman Coulter counter (model LS6000SC). The amount of [³H]—H₂O collected indicates the extent of fatty acid oxidation.

Western blot analysis. Tissue lysates were prepared in TPER buffer (Pierce) with phosphatase and protease inhibitors added (Calbiochem). Protein concentrations were determined using the Coomassie Plus protein assay reagent (Thermo Scientific). 10 μg of protein from tissue lysates or 1 μl serum were loaded and separated on a 10% Bis-Tris NuPAGE gel (Invitrogen). Western blotting and quantification were carried out as previously described (J Biol Chem 287: 10301-10315, 2012).

Statistical analysis. All results are expressed as mean±standard error of the mean (SEM). Statistical analysis was performed with Prism 5 software (GraphPad). Blood chemistry data were analyzed with two-tailed Student's t-tests between CTRP9 Tg and control littermates. Repeated measures ANOVA were performed on body weights as well as serum glucose and insulin measurements in various tolerance tests. Values were considered to be significant at p<0.05.

### Example 1

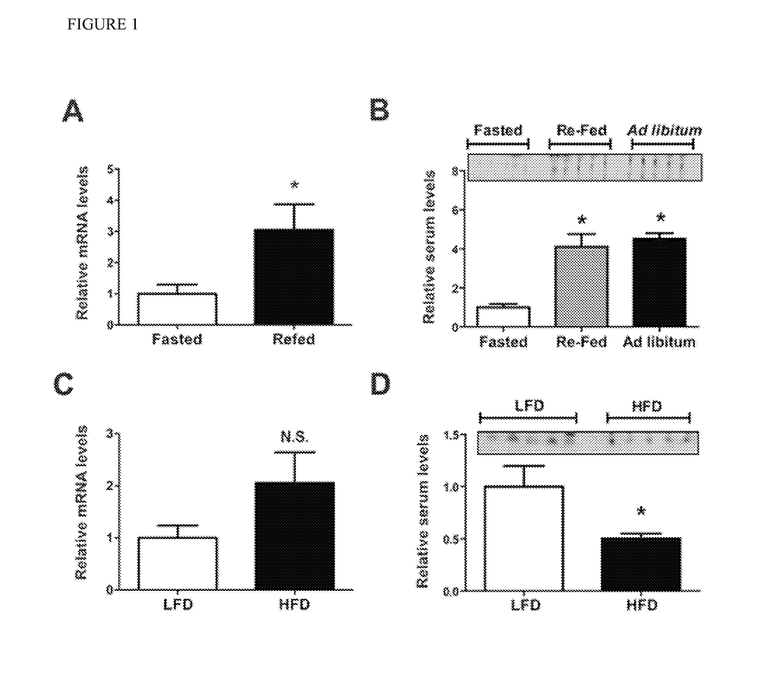

Metabolic state affects circulating levels of CTRP9. Expression and circulating levels of many adipokines change in response to alterations in energy state. It was therefore, first determined whether changes in nutritional states acutely affect circulating levels of CTRP9. Mice fed ad libitum and mice that were fasted/re-fed had 3-fold higher transcript levels of CTRP9 in adipose tissue and 4-fold higher circulating levels compared to overnight fasted animals (FIGS. 1A,B). In diet-induced obese male mice fed a high-fat diet (HFD) for 12 weeks, a ˜50% reduction in circulating levels of CTRP9 was observed (FIG. 1D). However, reduced serum levels of CTRP9 did not result from a reduction in mRNA expression in the adipose tissue (FIG. 1C). This suggests that diet-induced alteration in serum CTRP9 levels may be mediated by a posttranscriptional mechanism. Together, these data indicate that short-term changes in nutritional state, as well as chronic metabolic stress induced by a high fat-diet, alter circulating levels of CTRP9.

### Example 2

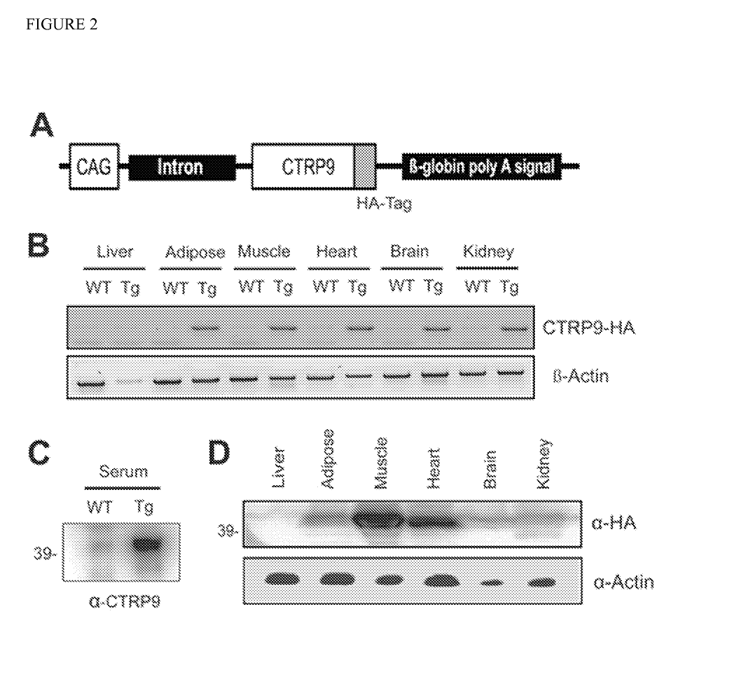

Generation of CTRP9 transgenic mice. To address the in vivo metabolic function of CTRP9 a transgenic (Tg) mouse model over-expressing HA epitope-tagged CTRP9 was generated. In mice, endogenous CTRP9 mRNA is expressed predominantly by adipose tissue, with lower expression levels in other tissues. Within adipose tissue, both adipocytes and cells of the stromal vascular fraction express CTRP9 mRNA. Therefore, expression of the CTRP9 transgene was driven by a ubiquitous promoter (FIG. 2A). In the Tg mouse line, CTRP9 expression was detected in the adipose tissue, skeletal muscle, heart, brain, and kidney, but was absent from the liver (FIG. 2B). Despite comparable expression of CTRP9 transgene mRNA in various mouse tissues, a substantially higher level of CTRP9-HA protein was detected in the skeletal muscle and heart compared to other tissues (FIG. 2D). As expected, over-expression also resulted in a significant increase in circulating levels of CTRP9, approximately 5-fold greater than baseline serum levels in wild-type mice (FIG. 2C). This gain-of-function mouse model enabled exploration of the long-term metabolic consequences of elevated circulating levels of CTRP9.

### Example 3

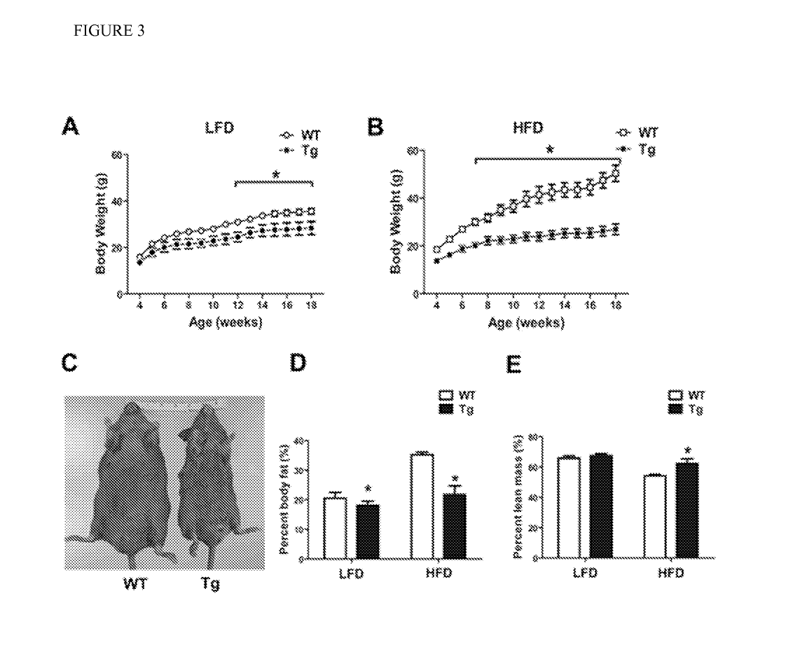

CTRP9 Tg mice are lean and resistant to diet-induced weight gain. Although comparable in body weight at weaning (4 weeks of age), Tg mice fed a low-fat diet (LFD) consistently gained less body weight over time (FIG. 3A). By 18 weeks of age, Tg mice fed a LFD were ˜22% lighter than control littermates. Remarkably, when fed an HFD, the body weight phenotype of the animals became overtly pronounced. While control littermates became progressively obese over time on an HFD as expected, Tg mice were resistant to weight gain (FIGS. 3B-C). In fact, by 18 weeks of age, Tg mice (26.9±2.4 g) were just half the body weight of control littermates (50.4±3.7 g). Strikingly, Tg mice on an HFD gained less body weight and accumulated similar amounts of fat mass (percent of body weight) compared to Tg mice on an LFD (FIGS. 3A-B). These data have been independently confirmed in three separate cohorts of Tg mice and their WT littermate controls. Thus, the beneficial and protective metabolic function of CTRP9 was revealed when mice were challenged with an HFD to induce metabolic stress.

### Example 4

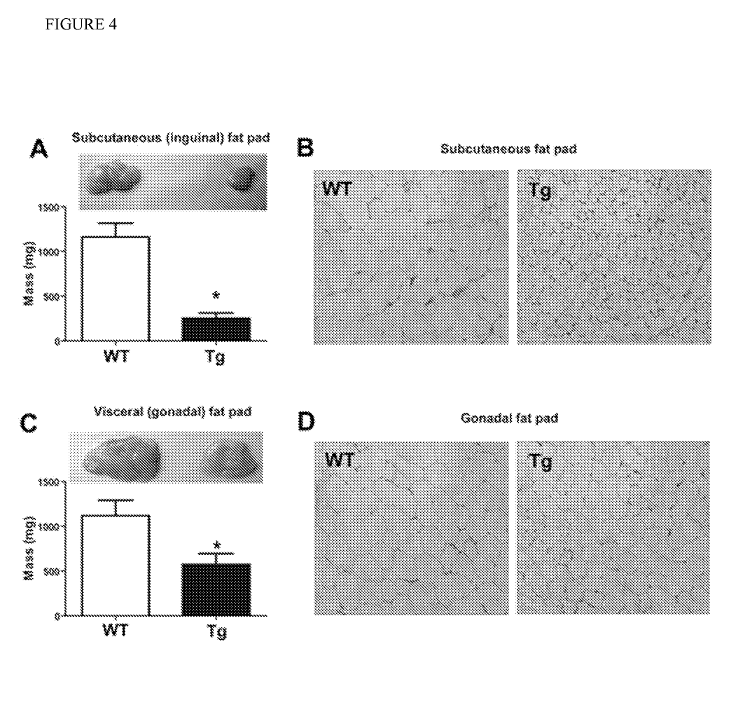

Reduced adiposity and adipocyte size in CTRP9 Tg mice. Differences in body weight between WT and Tg mice fed an HFD could result from reduced accumulation of fat mass. Indeed, body composition analysis using quantitative NMR revealed that Tg mice had significantly less fat mass compared to WT controls (FIG. 3D), accounting for the lower body weight of Tg mice. In contrast, an increase in percent lean mass (˜9%) in Tg mice fed an HFD relative to littermate controls was observed (FIG. 3E). Reduction in adiposity appeared more striking in subcutaneous (inguinal fat pad) compared to visceral fat depots (gonadal fat pad) in Tg mice relative to littermate controls (FIGS. 4A,C). Further, the size of adipocytes was significantly smaller in Tg mice (FIGS. 4B-D); again the difference was more pronounced in subcutaneous fat pad. The number of adipocytes in a given random 20× magnification field was quantified. In the gonadal fat pad, there were 79+14 (WT) vs. 183+27 (Tg) adipocytes per 20× field (n=9 for WT and n=6 for Tg mice). In the subcutaneous fat pad, there were 101+9 (WT) vs. 346+39 (Tg) adipocytes per 20× field (n=9 for WT and n=6 for Tg mice). Based on these data, it was estimated that the adipocytes found in the gonadal and subcutaneous fat depot of Tg mice are, on average, 2.3 and 3.4 times smaller than the WT counterparts, respectively. Smaller adipocytes are associated with improved metabolic profile and insulin sensitivity of white adipose tissue. We also looked for potential “browning” of the adipose compartment; no differences were observed in the mRNA levels of key marker genes (UCP-1, Elovl3, Otop1, Cox7a1) previously shown to be associated with brown adipocyte-like cells within white adipose tissue.

### Example 5

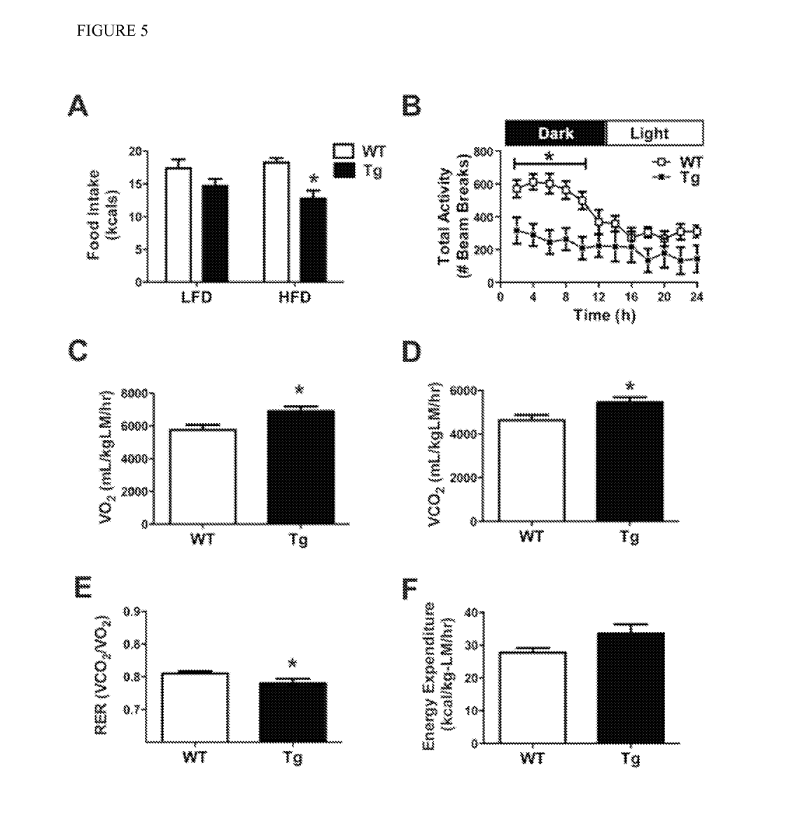

CTRP9 Tg mice show enhanced energy expenditure. Several mechanisms could account for the lean phenotype of CTRP9 Tg mice, such as differences in food intake, voluntary physical activity levels, and/or energy expenditure. On an LFD, WT and Tg mice consumed comparable amounts of food pellets (FIG. 5A). However, Tg mice fed an HFD consumed less calories (FIG. 5A). No differences in ambulatory activity were observed between WT and Tg mice fed an LFD (data not shown). In contrast, CTRP9 Tg mice on an HFD were physically less active during the dark photocycle compared to littermate controls (FIG. 5B).

Indirect calorimetry analyses were carried out to determine the consequences of CTRP9 over-expression on whole-body energy balance. On an LFD, no differences in VO₂, VCO₂, respiratory exchange ratio (RER), or energy expenditure were observed between WT and Tg mice (data not shown). In contrast, Tg mice fed an HFD had increased oxygen consumption (FIG. 5C) and increased carbon dioxide production (FIG. 5D) relative to littermate controls, indicating an enhanced metabolic rate. As expected, mice fed an HFD had lower respiratory exchange ratio (RER) compared to mice fed an LFD, due to a greater oxidation of lipid over carbohydrate substrates (data not shown). Within the HFD group, CTRP9 Tg mice had a lower RER compared to littermate controls (FIG. 5E), indicating greater fat oxidation. Due to increased metabolic rate, Tg mice fed an HFD had a modest overall increase in whole-body energy expenditure compared to WT mice (FIG. 5F). These data suggest that a combination of reduced food intake and increased basal metabolism over a 14-week period on HFD could account for the remarkable differences in body weight and percent fat and lean mass between WT and CTRP9 Tg mice (FIG. 3).

### Example 6

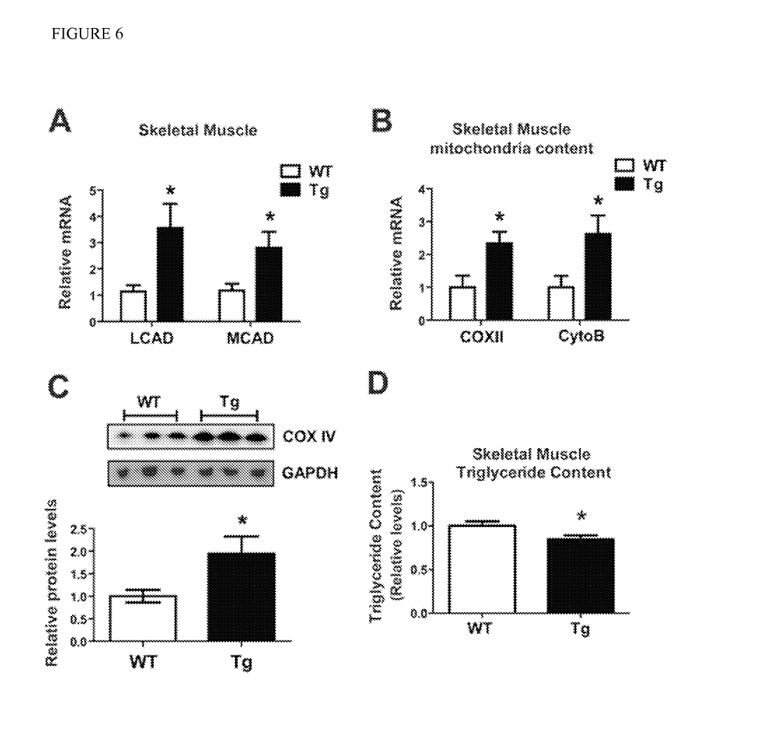

CTRP9 enhances AMPK activation and promotes skeletal muscle fat oxidation. To uncover the mechanism by which CTRP9 promotes energy expenditure, possible signaling pathways and enzymes that regulate fat oxidation in skeletal muscle were examined. Consistent with enhanced fat oxidation, a 3-fold increase in the expression of fatty acid oxidation enzyme genes (LCAD and MCAD) was observed in the skeletal muscle of Tg mice relative to control littermates (FIG. 6A). Further, there was over a 2-fold increase in the expression of mitochondrion-specific genes (COX II and CytoB) in the skeletal muscle of Tg mice (FIG. 6B). As with the mRNA expression data, mitochondrion-specific protein (e.g., COX IV) levels were also significantly increased (˜2-fold) (FIG. 6C), indicating greater mitochondrial content in the skeletal muscle of Tg mice on an HFD. Increased fat oxidation consequently resulted in lower triglyceride content in the skeletal muscle of Tg mice relative to control littermates fed an HFD (FIG. 6D).

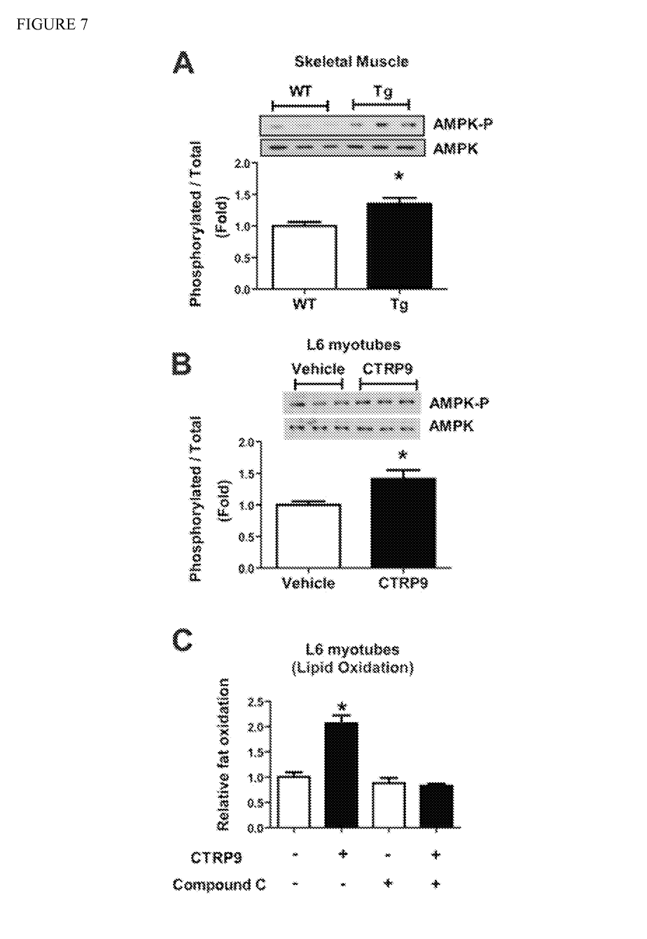

AMP-activated protein kinase (AMPK) phosphorylation (at Thr-172) and activation is known to increase mitochondrial biogenesis and muscle fat oxidation. In the skeletal muscle of CTRP9 Tg mice, AMPKα (the catalytic subunit) was hyperphosphorylated at Thr-172 relative to WT controls (FIG. 7A), indicating enhanced AMPK activation. In accordance with a direct effect of CTRP9 in promoting muscle fat oxidation in vivo, purified recombinant CTRP9 likewise induced AMPKα (Thr-172) phosphorylation (FIG. 7B) and enhanced fatty acid oxidation in differentiated rat L6 myotubes (FIG. 7C). Treatment of myotubes with compound C, an AMPK inhibitor, abolished the ability of CTRP9 to enhance fatty acid oxidation (FIG. 7C), confirming that CTRP9 activates AMPK signaling to control fat oxidation. Together, these data indicate that CTRP9 promotes energy expenditure in vivo by enhancing skeletal muscle fat oxidation via AMPK activation and by increasing mitochondrial content.

It has been increasingly appreciated that brown adipose tissue also plays an important role in whole-body energy expenditure by promoting fat oxidation. While the white adipose tissue mass was greatly reduced in Tg mice (FIG. 3D), there was no observation of any changes in interscapular brown adipose tissue mass (normalized to body weight) nor any differences in the expression levels of uncoupling protein-1 (UCP1) mRNA and protein in brown fat isolated from WT and Tg mice fed an HFD (data not shown), indicating minimal contribution by brown fat to overall differences in energy expenditure observed between WT and Tg mice.

### Example 7

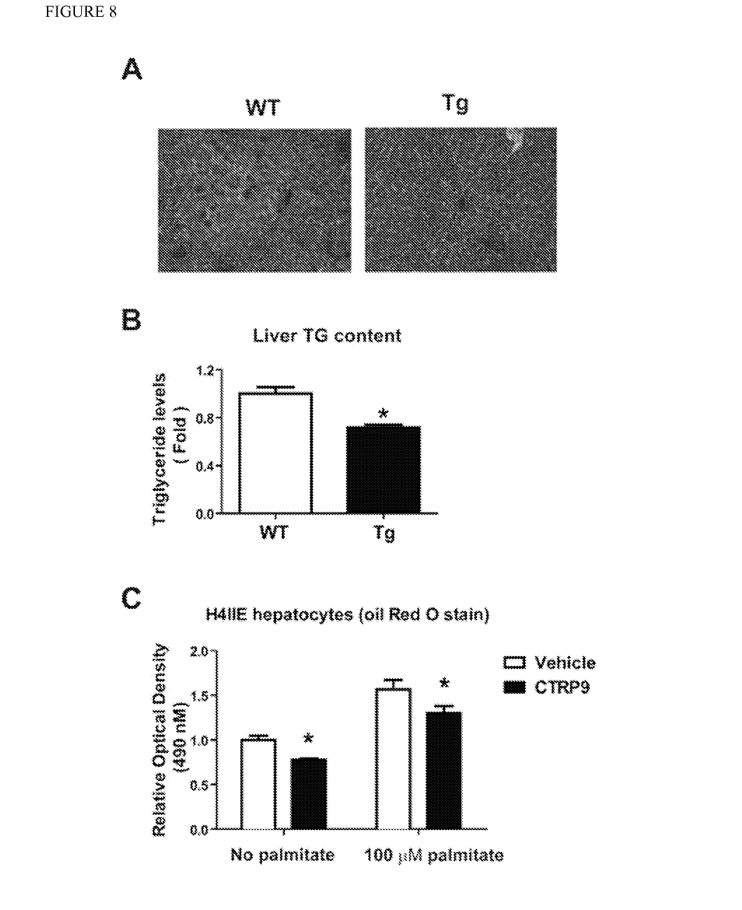

CTRP9 decreases hepatic lipid accumulation. While HFD leads to hepatic triglyceride accumulation and, consequently, fatty liver (hepatic steatosis) in WT mice, over-expressing CTRP9 substantially reduced hepatic triglyceride levels by ˜30% (FIG. 8A,B). Circulating ketone levels, a product of hepatic fatty acid oxidation, were elevated in Tg mice relative to WT controls (Table 1), indicating enhanced fat oxidation in the liver. This may account, in part, for lower hepatic triglyceride content in Tg mice. Consistent with the in vivo data, recombinant CTRP9 treatment reduced basal as well as palmitate-induced lipid accumulation in cultured H4IIE hepatocytes (FIG. 8C). Together, these results suggest that skeletal muscle and liver are target tissues of CTRP9 in vivo and are likely responsible for oxidizing a substantial amount of the available lipid substrates.

TABLE 1
Blood chemistry analysis. Sera were collected from
overnight-fasted WT and Tg mice (n = 8-10)
when they were 18 weeks old and had been on HFD
for the previous 14 weeks. Values are means ± SEM.

| | WT | Tg | P-Value |
|---|---|---|---|
| Glucose (mg/dL) | 151 ± 12.1 | 86 ± 6.9 | <0.05 |
| Insulin (ng/mL) | 1.73 ± 0.4 | 0.58 ± 0.1 | <0.05 |
| Glucagon (pg/mL) | 18.4 ± 3.4 | 14.6 ± 2.4 | <0.05 |
| Leptin (ng/mL) | 13.65 ± 2.4 | 4.54 ± 1.6 | <0.05 |
| Adiponectin (μg/mL) | 11.9 ± 2.4 | 12.9 ± 2.8 | ns |
| Ketones (mM) | 1.52 ± 0.14 | 2.1 ± 0.12 | <0.05 |
| NEFA (mEq/L) | 0.99 ± 0.07 | 1.36 ± 0.18 | ns |
| Serum Triglycerides (mg/dL) | 72.1 ± 4.1 | 94.7 ± 8.7 | <0.05 |

### Example 8

Improved metabolic profiles of mice over-expressing CTRP9. Enhanced fat oxidation coupled with reduced hepatic and skeletal muscle triglyceride levels are predicted to improve the metabolic profiles of CTRP9 Tg mice. Indeed, on an HFD, overnight-fasted Tg mice had markedly reduced insulin and glucose levels compared to littermate controls (Table 1), indicating enhanced insulin sensitivity in Tg animals. As expected from a reduction in fat mass, serum leptin levels in Tg mice were correspondingly decreased relative to WT controls. Serum glucagon levels were also reduced in Tg mice, likely a consequence of decreased insulin levels. On an HFD, Tg mice have higher circulating levels of triglycerides and no significant difference in non-esterified free fatty acid (NEFA). Circulating levels of adiponectin were comparable between WT and Tg mice.

### Example 9

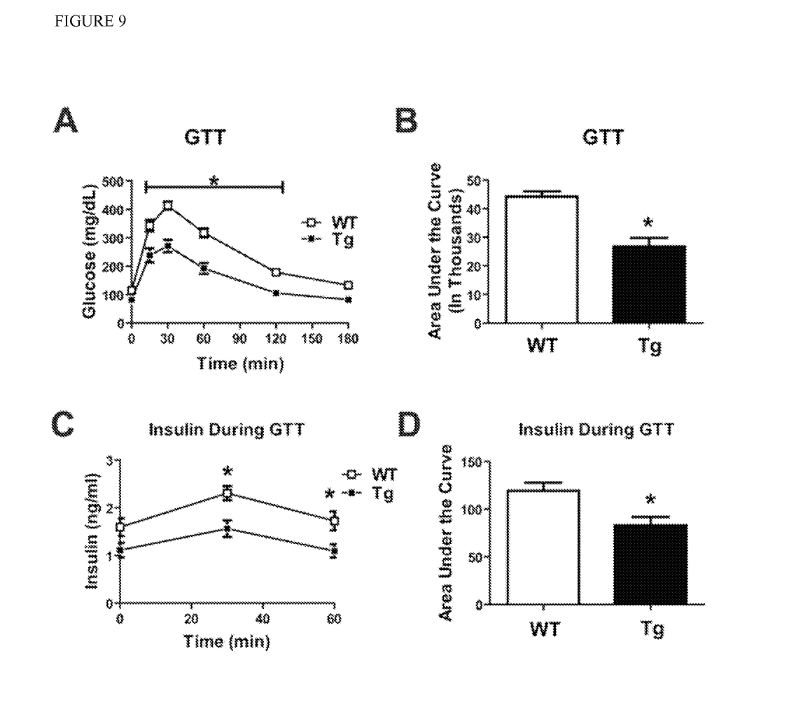

CTRP9 prevents HFD-induced insulin resistance. Assessment of whole-body insulin sensitivity using the homeostatic model assessment insulin resistance index (HOMA-IR) revealed a substantial reduction in insulin resistance in CTRP9 Tg mice relative to WT controls fed an HFD (data not shown). To confirm that Tg mice were indeed more insulin sensitive, an intraperitoneal glucose tolerance test (GTT) was performed. Mice over-expressing CTRP9 were much better at handling a glucose load, with a significantly higher rate of glucose disposal in the peripheral tissues (FIG. 9A-B). The rate of glucose excursion in GTT is dependent on the magnitude of insulin secretion from the pancreatic β cells in response to glucose challenge, as well as peripheral tissue insulin sensitivity. Therefore, insulin levels were measured. The magnitude of insulin secretion in Tg mice in response to the GTT was significantly lower (FIG. 9C-D) despite a much greater rate of glucose disposal in these animals. Together, these results indicate that CTRP9 Tg mice on an HFD are indeed more insulin sensitive compared to littermate controls.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1-4. (canceled)

5. A method for identifying a molecule which increases CTRP9 protein levels in a cell or population of cells capable of expressing the CTRP9 protein comprising:
a) obtaining a cell or population of cells which express CTRP9 protein;
b) incubating the molecule with the cell or population of cells of a);
c) measuring the levels of CTRP9 expression, in the cell or population of cells of a);
d) comparing the levels of CTRP9 in the cell or population of cells of a) to that of a control cell or population of cells; and
e) determining that the molecule increases CTRP9 protein levels in a cell or population of cells when the protein levels of CTRP9 are greater than the control cell or population of cells.

6. The method of claim 5, wherein the cell or population of cells is a myocyte.

7. A method for treating obesity and/or Type II diabetes in a subject comprising administering to the subject an effective amount of CTRP9 protein or a functional portion thereof, in a pharmaceutically acceptable carrier.

8. A method for increasing the levels of fatty acid oxidation in the skeletal muscle of a subject comprising administering to the subject an effective amount of CTRP9 protein or a functional portion thereof, in a pharmaceutically acceptable carrier.

9. A method for decreasing hepatic lipid accumulation in a subject comprising administering to the subject an effective amount of CTRP9 protein or a functional portion thereof, in a pharmaceutically acceptable carrier.

10. The method of claim 7, wherein the method further comprises administration of at least one additional active ingredient.

11. The method of claim 10, wherein the at least one addition active ingredient is selected from the group consisting of dipeptidyl peptidase IV (DP-IV) inhibitors, insulin sensitizers including PPARγ agonists, PPARα/γ dual agonists, PPARα agonists, biguanides, protein tyrosine phosphatase-1B (PTP-1B) inhibitors, insulin or insulin mimetics, sulfonylureas and other insulin secretagogues, α-glucosidase inhibitors, glucagon receptor antagonists, GLP-1, GLP-1 mimetics, and GLP-1 receptor agonists, GIP and GIP mimetics, PACAP, PACAP mimetics, and PACAP receptor 3 agonists, cholesterol lowering agents, sequestrants, nicotinyl alcohol, nicotinic acid or a salt thereof, inhibitors of cholesterol absorption, acyl CoA:cholesterol acyltransferase inhibitors, anti-oxidants, antiobesity compounds, β3 adrenergic receptor agonists; ileal bile acid transporter inhibitor, and agents intended for use in inflammatory conditions such as aspirin, non-steroidal anti-inflammatory drugs, glucocorticoids, azulfidine, and cyclo-oxygenase 2 selective inhibitors.

12. The method of claim 8, wherein the method further comprises administration of at least one additional active ingredient.

13. The method of claim 12, wherein the at least one addition active ingredient is selected from the group consisting of dipeptidyl peptidase IV (DP-IV) inhibitors, insulin sensitizers including PPARγ agonists, PPARα/γ dual agonists, PPARα agonists, biguanides, protein tyrosine phosphatase-1B (PTP-1B) inhibitors, insulin or insulin mimetics, sulfonylureas and other insulin secretagogues, α-glucosidase inhibitors, glucagon receptor antagonists, GLP-1, GLP-1 mimetics, and GLP-1 receptor agonists, GIP and GIP mimetics, PACAP, PACAP mimetics, and PACAP receptor 3 agonists, cholesterol lowering agents, sequestrants, nicotinyl alcohol, nicotinic acid or a salt thereof, inhibitors of cholesterol absorption, acyl CoA:cholesterol acyltransferase inhibitors, anti-oxidants, antiobesity compounds, β3 adrenergic receptor agonists; ileal bile acid transporter inhibitor, and agents intended for use in inflammatory conditions such as aspirin, non-steroidal anti-inflammatory drugs, glucocorticoids, azulfidine, and cyclo-oxygenase 2 selective inhibitors.

14. The method of claim 9, wherein the method further comprises administration of at least one additional active ingredient.

15. The method of claim 14, wherein the at least one addition active ingredient is selected from the group consisting of dipeptidyl peptidase IV (DP-IV) inhibitors, insulin sensitizers including PPARγ agonists, PPARα/γ dual agonists, PPARα agonists, biguanides, protein tyrosine phosphatase-1B (PTP-1B) inhibitors, insulin or insulin mimetics, sulfonylureas and other insulin secretagogues, α-glucosidase inhibitors, glucagon receptor antagonists, GLP-1, GLP-1 mimetics, and GLP-1 receptor agonists, GIP and GIP mimetics, PACAP, PACAP mimetics, and PACAP receptor 3 agonists, cholesterol lowering agents, sequestrants, nicotinyl alcohol, nicotinic acid or a salt thereof, inhibitors of cholesterol absorption, acyl CoA:cholesterol acyltransferase inhibitors, anti-oxidants, antiobesity compounds, β3 adrenergic receptor agonists; ileal bile acid transporter inhibitor, and agents intended for use in inflammatory conditions such as aspirin, non-steroidal anti-inflammatory drugs, glucocorticoids, azulfidine, and cyclo-oxygenase 2 selective inhibitors.

16. A method for prevention of diet induced insulin resistance in a subject comprising administering to the subject an effective amount of CTRP9 protein or a functional portion thereof, in a pharmaceutically acceptable carrier.

17. The method of claim 16, wherein the method further comprises administration of at least one additional active ingredient.

18. The method of claim 17, wherein the at least one addition active ingredient is selected from the group consisting of dipeptidyl peptidase IV (DP-IV) inhibitors, insulin sensitizers including PPARγ agonists, PPARα/γ dual agonists, PPARα agonists, biguanides, protein tyrosine phosphatase-1B (PTP-1B) inhibitors, insulin or insulin mimetics, sulfonylureas and other insulin secretagogues, α-glucosidase inhibitors, glucagon receptor antagonists, GLP-1, GLP-1 mimetics, and GLP-1 receptor agonists, GIP and GIP mimetics, PACAP, PACAP mimetics, and PACAP receptor 3 agonists, cholesterol lowering agents, sequestrants, nicotinyl alcohol, nicotinic acid or a salt thereof, inhibitors of cholesterol absorption, acyl CoA:cholesterol acyltransferase inhibitors, anti-oxidants, antiobesity compounds, β3 adrenergic receptor agonists; ileal bile acid transporter inhibitor, and agents intended for use in inflammatory conditions such as aspirin, non-steroidal anti-inflammatory drugs, glucocorticoids, azulfidine, and cyclo-oxygenase 2 selective inhibitors.
